(19) United States
(12) Patent Application Publication
(10) Pub. No.: US 2018/0169340 A1
(43) Pub. Date: Jun. 21, 2018

(54) METHOD FOR PRODUCING AT LEAST ONE RESERVOIR ASSEMBLY, INJECTABLE-PRODUCT RESERVOIR ASSEMBLY FOR AN INJECTION PEN AND INJECTION PEN EQUIPPED WITH SUCH AN ASSEMBLY

(71) Applicant: BIOCORP PRODUCTION, Issoire (FR)

(72) Inventor: Antoine Aneas, Menetrol (FR)

(21) Appl. No.: 15/579,113

(22) PCT Filed: Jun. 1, 2016
(86) PCT No.: PCT/EP2016/062394

§ 371 Date: Dec. 1, 2017

(30) Foreign Application Priority Data
Jun. 2, 2015 (FR) 1554991

(51) Int. Cl. A61M 5/24

(52) U.S. Cl. CPC A61M 5/24; A61M 2005/2437; A61M 2005/2407

(57) ABSTRACT

The method is used to produce at least one injectable-product reservoir assembly for an injection pen. The method comprises at least the sequence of steps consisting of: producing a hollow housing equipped with a first mechanical member for attaching a needle and a second mechanical member for attaching the housing onto an injection pen; inserting (F6) an elongate container having an open bottom into the housing; and filling the container with a predetermined quantity of injectable product, through an opening of the open bottom of the container.

The invention relates to a method for producing at least one assembly forming an injectable product reservoir for an injection pen. The invention also relates to an assembly that forms an injectable product reservoir for an injection pen, as well as an injection pen equipped with such an assembly.

The invention belongs to the field of injection pens used to inject an active ingredient parenterally, i.e., intravenously, subcutaneously or intramuscularly, into a patient's body. Such a pen can be used to treat a patient with a specific disease, such as diabetes or dwarfism. Indeed, the treatment of such a patient requires at least one injection per day, of insulin in the case of diabetes or growth hormone in the case of dwarfism. In light of the number of injections needed, patients often perform their injections themselves, without the assistance of a healthcare provider.

To that end, patients increasingly frequently use an injection pen, which is easier to use than a traditional syringe, since it can be stored discreetly and safely. An injection pen is implemented with containers, most often called “cartridges”, with relatively small holding capacities and nevertheless making it possible to perform a certain number of injections. A cartridge is a cylindrical container with a circular section, the length of which is about 60 mm and the outer diameter of which is comprised between 8.5 and 12 mm, preferably about 11 mm. Such an injection pen is easy for the patient to transport, like a traditional pen.

To use such an injection pen, the latter must be equipped with a container containing the active ingredient to be injected, namely a capsule or cartridge as set out above. Before each use, a needle must be mounted at the distal end of the container, this needle being thrown away after use, while the container remains on the injection pen. The container should therefore be kept in position on a base of the injection pen.

To that end, it is known from US-A-2014/0358093 to provide, at the front of the base of an injection pen, a cartridge support suitable for receiving a cartridge full of active ingredient to be injected. The immobilization of each cartridge in this support takes place using a coupling element that is mounted around one end of the cartridge and that is provided to cooperate with the cartridge support. This coupling element leaves the majority of the cartridge exposed, while the latter is not mounted within the injection pen, resulting in a non-negligible risk of this cartridge breaking, the contents of which are then lost, which prevents treatment.

It is furthermore known from WO-A-2013/124118 to use jaws arranged at the end of flexible arms to retain a cartridge of active ingredient inside a support of an injection pen. This equipment is relatively complex to use, in particular by a patient who may be weakened or unfamiliar with these manipulations.

Furthermore, DE-A-10 2013 007 389 teaches inserting, into a hollow housing, a container that is already filled and closed off by a piston and jamming this container in place using a retaining ring inserted into the hollow housing after the container. The sustainability of the assembly of the container and the housing depends on the machining allowances of the container of the housing and the retaining ring. If the container is too short, it risks sliding in the housing. If it is too long, the retaining ring cannot be placed.

In the known equipment, in particular from DE-A-10 2013 007 389, the cartridge must be equipped with its piston, then filled through its throat, before being equipped with a stopper device and an identification sticker. Between the production of the cartridge and the filling/plugging/marking thereof, the cartridge must generally go from the production site of a glass worker to the packaging site for the active ingredient, generally within a pharmaceutical laboratory. During this transit, the cartridge is open at both ends. When the cartridge is received on its filling site, it must be washed, dried, coated with a lubricant, for example silicone, to allow its piston to slide, then sterilized. Next, the piston must be inserted into the cartridge before the latter is turned over to be filled through its throat, in an aseptic environment. Later, a stopper and an aluminum cap are optionally attached on the cartridge, then the cap is crimped, still in an aseptic environment. These operations are complex and consume considerable time, space and resources to manipulate each cartridge. Furthermore, the crimping of the cap generates aluminum, glass, paint and/or varnish particles that may pollute the aseptic environment.

The invention more particularly aims to resolve these drawbacks by proposing a new method for manufacturing at least one assembly forming a reservoir for an injectable product that is more reliable and more practical to implement than the known methods.

To that end, the invention relates to a method for producing at least one assembly forming an injectable product reservoir for an injection pen. This method comprises at least the following successive steps:

a) producing a hollow housing equipped with a first mechanical member for attaching a needle and a second mechanical member for attaching the housing onto an injection pen;

b) inserting, in an inner volume of the housing, an elongated container with an open bottom;

c) filling the container with a predetermined quantity of injectable product, through an opening of the open bottom of the container.

Steps a) to c) take place one after another, in this order. Owing to the invention, the elongated container, of the cartridge type, may be protected by the housing when it is inserted therein, then filled with injectable product once it is in place in the housing, which facilitates the packaging of the injectable product.

According to advantageous but optional aspects of the invention, such a housing may incorporate one or more of the following features, considered in any technically allowable combination:

During step b), the insertion of the container into the hollow housing takes place through an open end of the body of this housing near which the second mechanical member is positioned for fastening the housing on an injection pen.

Prior to step b), a longitudinal axis of the container is aligned with a longitudinal axis of the hollow housing and, during step b), the insertion of the container into the housing takes place by a translation of the container relative to the hollow housing along these two longitudinal axes.

During step b), the container is moved inside the housing until a flange of the container is axially immobilized, irreversibly, by at least one third mechanical locking member positioned in the vicinity of the first mechanical member for fastening a needle.

The third locking member is an elastically deformable tab of the housing, whereas, during step b), the flange of the container radially pushes the tab back, which deforms elastically, and when the flange has axially exceeded the tab, the latter regains its original configuration, as a result of which one edge of the tab, oriented opposite an open end of a body of the hollow housing, arrives across from, or bearing against, an annular surface of the flange turned toward one end of a body of the container opposite a throat of this container.

The method comprises a step d) after step a) and before step b) consisting of inserting a stopper closing a throat of the container into the housing.

During step b), the container is fully inserted into the inner volume of the housing.

The method comprises a step e), after step b) and before step c), consisting of positioning a draft product, formed from the housing equipped with the container, on a support, with the open bottom of the container oriented upward.

The method comprises the following additional steps:
f) before or after step e), positioning the support in a container,
g) closing the container using a closure member,
h) after step g), sterilizing the draft product.

At the end of step f), the housing extends at a nonzero distance above the bottom of the container.

The method comprises steps after step b) and before step c) for washing, drying and/or inspecting the draft product.

Steps a), b) and optionally d), e), f), g) and/or h) are carried out on a same industrial site, in particular a manufacturer of the container and/or the housing.

The support is equipped with several recesses, each with a shape complementary to the outer shape of a housing, and step f) is carried out successively, in order to have a housing equipped with a container in each recess.

The method comprises additional steps i) and j) after step c) and consisting of:
i) closing off the open bottom of the container with a piston inserted into an inner volume of this container,
j) identifying each reservoir-forming assembly for an injectable product by affixing a sticker or a marking on an outer surface of the housing,
   while steps c), i) and j) are carried out on a same industrial site, in particular within a pharmaceutical laboratory or on a drug packaging site.

Furthermore, the invention relates to an assembly forming an injectable product reservoir for an injection pen, this assembly comprising an elongated container containing an injectable product. This assembly further comprises a housing, the body of which defines an inner volume in which the elongated container is fully housed, while the body is provided, on the outside of a first axial end, with at least one first mechanical member for attaching a needle and, in the vicinity of a second axial end opposite the first axial end, with at least one second mechanical member for attaching the reservoir-forming assembly onto an injection pen, while the housing comprises, in the vicinity of the first axial end, at least one third mechanical member for irreversible axial locking of the container in a position fully contained in the inner volume of the body. Furthermore, the third member is an elastically deformable tab of the housing, which is intended to ensure the locking of the container in position in the inner volume of the body of the housing, by axially retaining a flange of the container.

Such an assembly can be manipulated in a unitary manner, in particular for transport and mounting on an injection pen, while the container is effectively protected from impacts and risks of pollution due to the fact that it is fully housed in the inner volume of the body of the housing.

Lastly, the invention relates to an injection pen, intended to be used to inject an active ingredient into the body of a patient and which comprises a base intended to be held by a user, as well as a removable injectable product reservoir. According to the invention, this injectable product reservoir is formed by an assembly as mentioned above, while the second mechanical member of the body of the housing is engaged with a complementary mechanical member arranged on the base.

The invention will be better understood, and other advantages thereof will appear more clearly, in light of the following description of two embodiments of a housing, an assembly forming a reservoir and an injection pen according to its principle, provided solely as an example and done in reference to the appended drawings, in which:

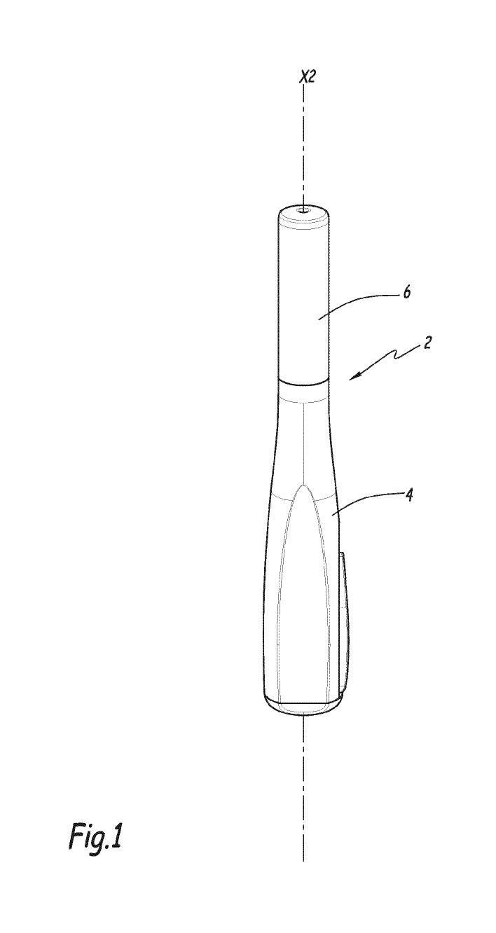

FIG. 1 is a perspective view of an injection pen according to the invention, in a storage or transport configuration;

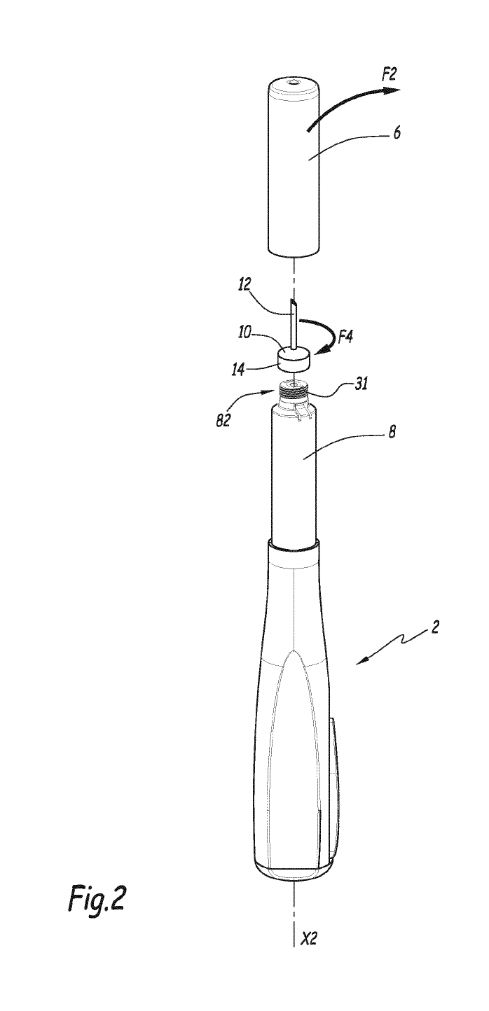

FIG. 2 is a perspective view of the pen of FIG. 1 in a pre-use configuration;

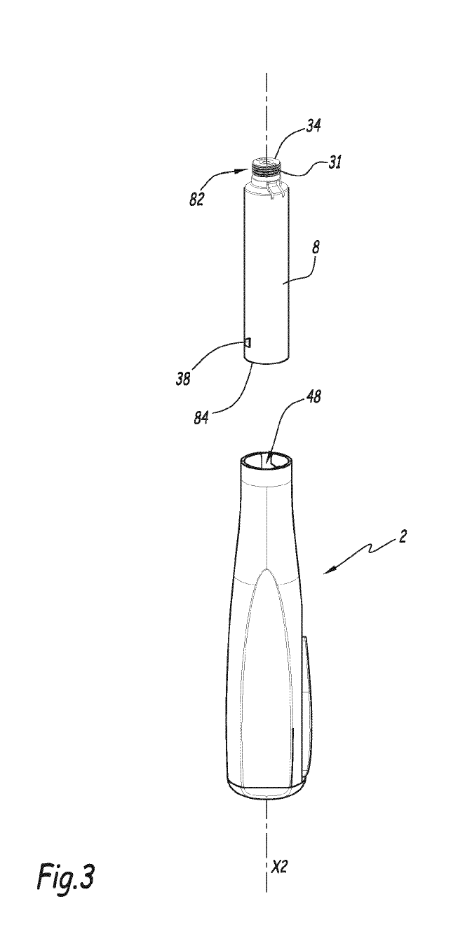

FIG. 3 is a perspective view similar to FIG. 1 in a configuration for changing an assembly forming a reservoir of injectable product for the injection pen of FIGS. 1 and 2;

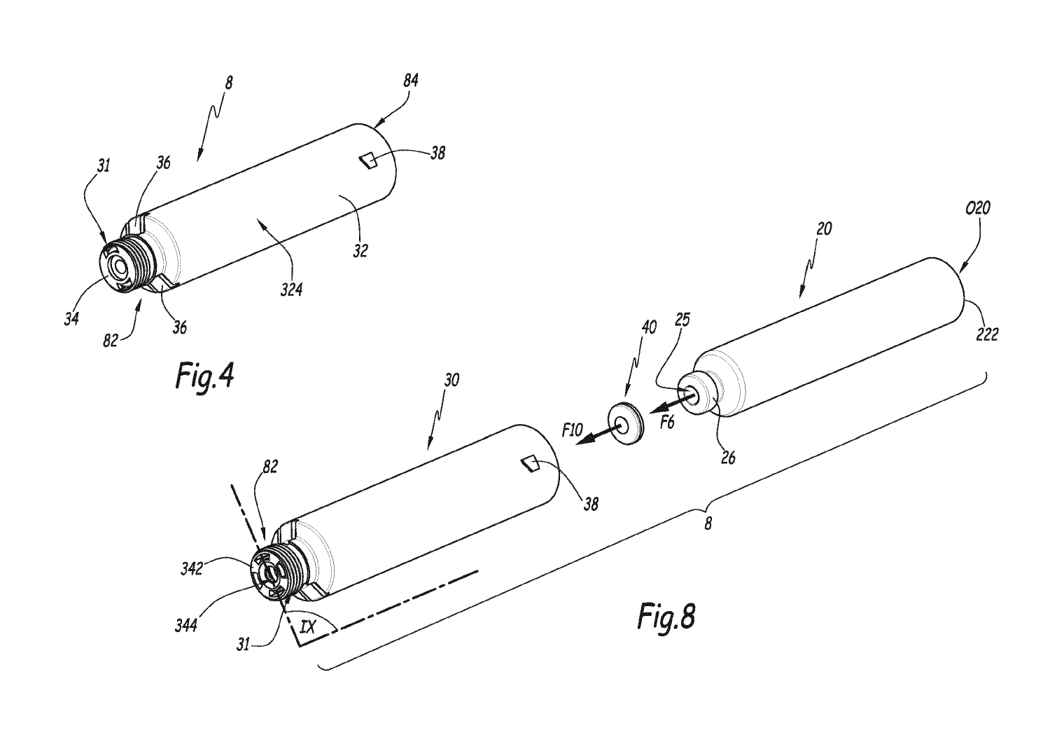

FIG. 4 is a perspective view, from another angle, of the reservoir-forming assembly visible in FIGS. 2 and 3;

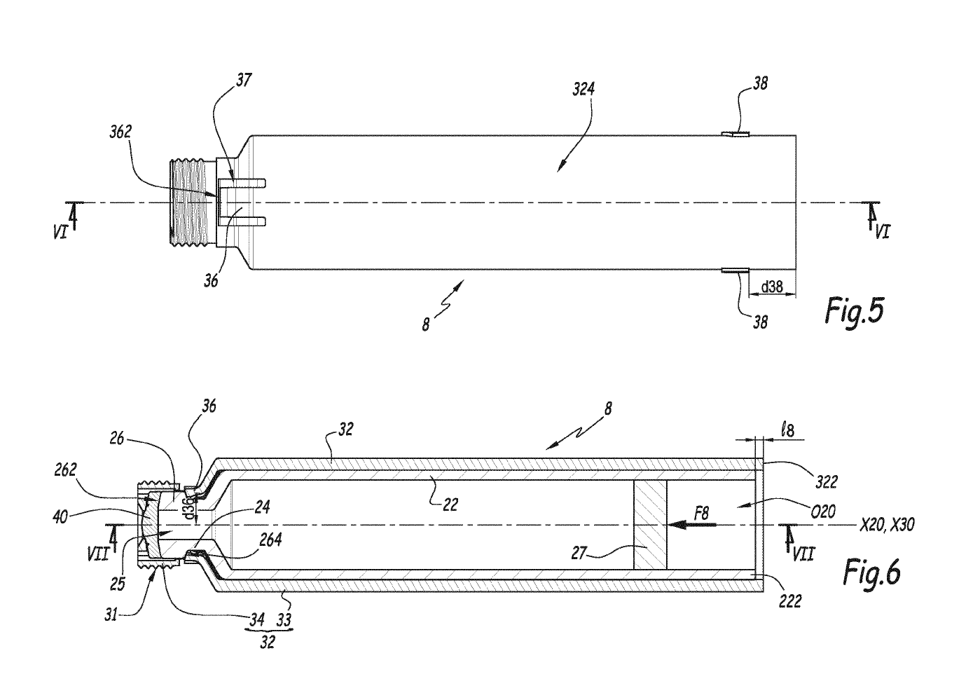

FIG. 5 is a side view of the reservoir-forming assembly of FIG. 4;

FIG. 6 is a sectional view along line VI-VI in FIG. 5;

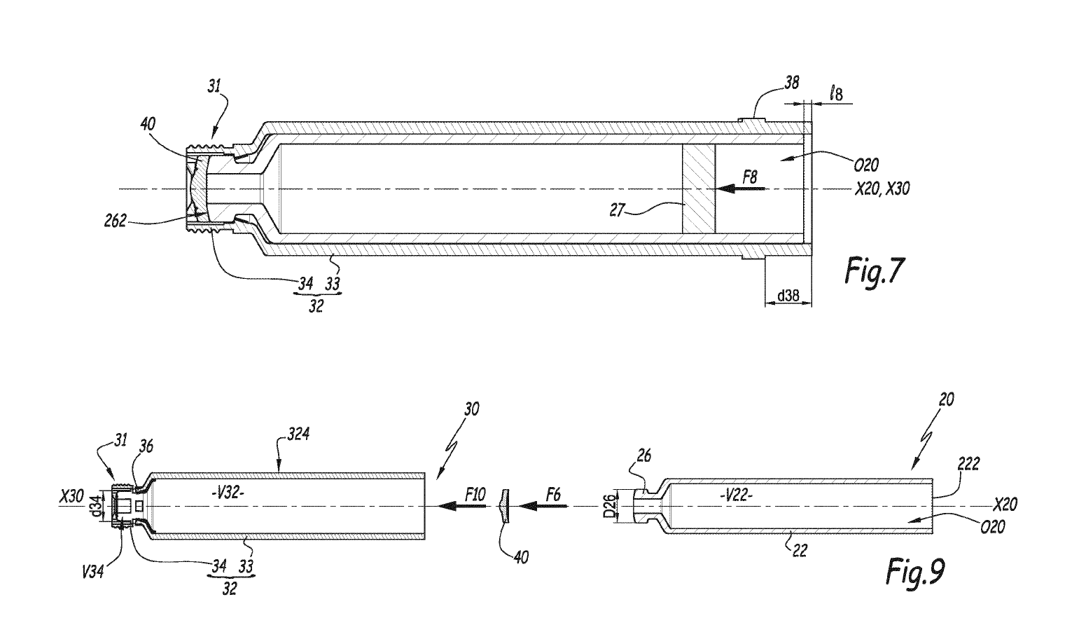

FIG. 7 is a sectional view along line VII-VII in FIG. 6;

FIG. 8 is an exploded perspective view of the reservoir-forming assembly during assembly;

FIG. 9 is a sectional view along plane IX in FIG. 8, on a smaller scale than FIGS. 6 and 7;

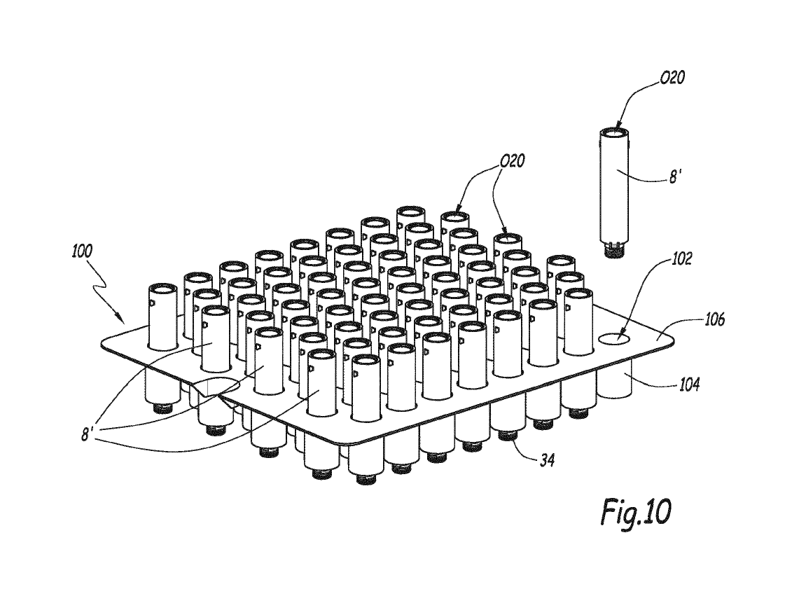

FIG. 10 is an exploded perspective view of a group of reservoir-forming assemblies, during production, after the step shown in FIGS. 8 and 9;

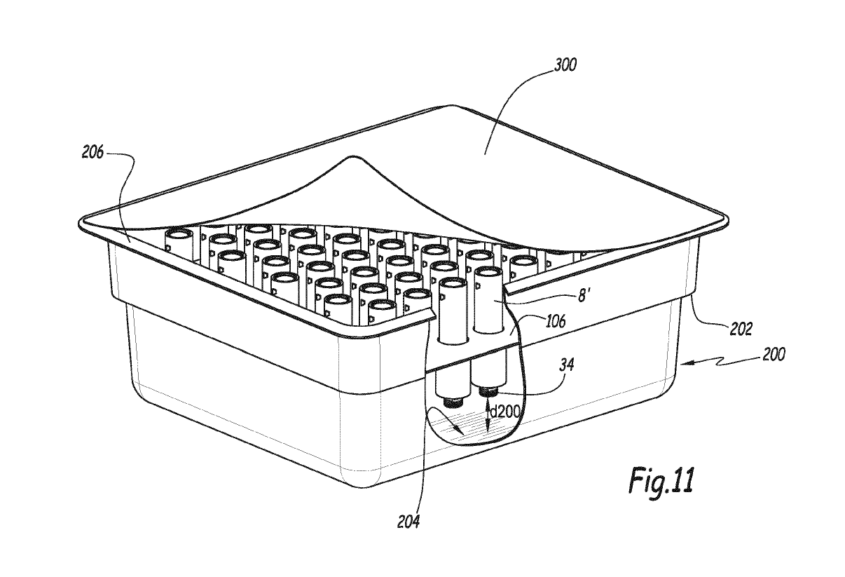

FIG. 11 is a perspective view corresponding to FIG. 10, during a following step of the production method; and

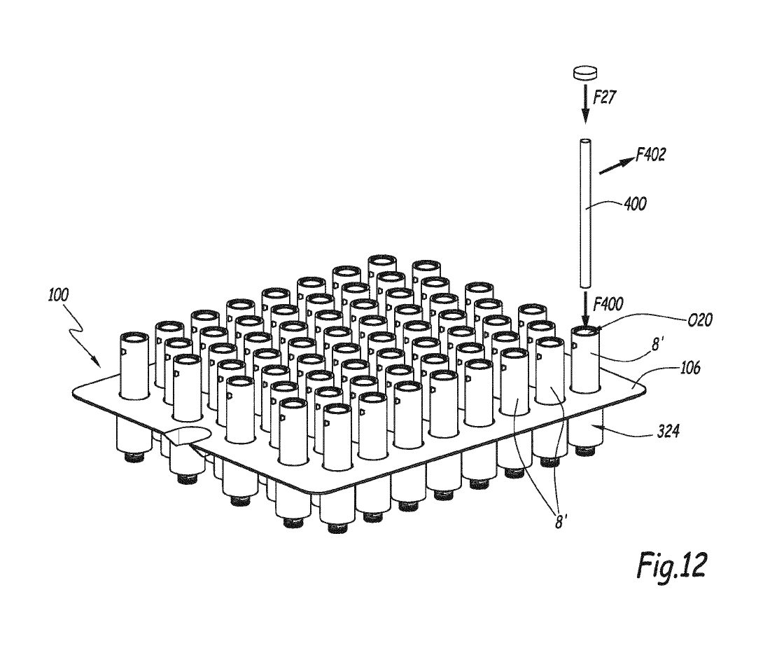

FIG. 12 is a perspective view corresponding to FIG. 10, during another following step of the production method according to the invention.

The injection pen 2 visible in FIGS. 1 to 3 extends along a longitudinal axis X2 and comprises a base 4, intended to be held by a user during an injection, as well as a cover 6 attached on the base 4 and which, when it is in place on this base 4, isolates a reservoir-forming assembly 8 from the outside, the latter in turn being mounted on the base 4, as shown by the following explanations.

The assembly 8 contains an injectable product, in particular a drug containing an active ingredient intended to treat a disease requiring treatment by regular doses, such as diabetes or dwarfism.

As shown in FIG. 2, when the injection pen 2 needs to be used, the cover 8 is removed, which is shown by arrow F2, and a disposable element 10, comprising the needle 12, is mounted at the distal end 82 of the assembly 8, which is shown by arrow F4. To that end, the distal end 82 is provided with an outer thread 31, while a base part 14 of the assembly 10 is equipped with an inner tapping with a corresponding geometry, not visible in FIG. 2. Thus, a disposable element 10 can be screwed on the distal end 82 before each use of the injection pen 2, its needle 12 then being aligned on the axis X2.

The reservoir-forming assembly 8 is shown alone in FIGS. 4 to 9. Two elements are formed, i.e., a carpule or cartridge 20 and a housing or enclosure 30 that is provided on the one hand to contain the cartridge 20, and on the other hand to allow, at the distal end 82, fastening thereof with the disposable element 10, and a proximal end 84 of the assembly 8, fastening thereof on the base 4.

The cartridge 20 is made from glass and forms an elongated container centered on a longitudinal axis X20 and having a cylindrical body 22 with a circular section, the outer diameter D22 of which is comprised between 8.55 and 12 mm. Alternatively, the cartridge 20 may be made from plastic. This cartridge is provided with a neck 24 defining a throat 25 through which it is possible to fill the cartridge or to remove an injectable product therefrom and that is surrounded by a flange 26, for which reference 262 denotes an annular surface surrounding the outlet of the throat 25 and reference 264 denotes an annular surface oriented away from the surface 262 and turned toward an end 222 of the body 22 opposite the throat 25.

A stopper 40 is used to close off the throat 25 while bearing against the surface 262.

Hereinafter, a direction is said to be axial when it is parallel to an axis and radial when it is perpendicular and secant to said axis. A surface is said to be axial when it is perpendicular to an axial direction and radial when it is perpendicular to a radial direction.

The surfaces 262 and 264 are axial surfaces relative to the axis X20.

The end 222 of the body 22 is open and defines an opening O20 centered on the axis X20. A piston 27 is housed in the body 22, through this opening O20, with a possibility of axial sliding along the axis X20. The axial sliding of the piston 27 in the body 22 is commanded by means integrated into the base 4, which are known in themselves and are not described here in detail.

The housing 30 is centered on a longitudinal axis X30 and comprises a body 32 made by molding plastic, for example polycarbonate.

The body 32 defines an inner volume V32 in which the cartridge 20 can be fully received. In other words, the cartridge 20 can be housed in the volume V32 without protruding outside the body 32 of the housing 30.

The body 32 comprises a main part 33, the inner diameter d33 of which is slightly larger than the diameter D22. Slightly larger means that the diameter d33 is less than 110% of the diameter D22. The body 32 also comprises a head 34, in one piece with the main part 33 and intended to receive the flange 26. The head 34 forms the distal end 82 of the assembly 8.

At the junction between the main part 33 of the body 32 and the head 34, the housing 30 is provided with two elastically deformable tabs 36 that are also in one piece with the rest of the body 32. The tabs 36 are each surrounded by a U-shaped cutout 37 with a flat bottom.

Reference 322 denotes the open end of the body 32 opposite the head 84, and reference 362 denotes the edge of each tab 36 oriented opposite the end 322. The end 32 forms the proximal end 84 of the assembly 8.

The body 32 has no window or opening. In other words, the body 32 is continuous around the axis X30, such that it completely isolates the volume V32 from the outside, except at the open end 322.

The head 34 is provided with a bottom 342 that defines a central opening 344 centered on the axis X30 and that provides access to the stopper 40 in the configuration with the cartridge 20 mounted in the housing 30. The inner diameter d34 of the head 34, defined in a part of this head 34 comprised between the bottom 342 and the edges 362 of the tabs 36, is slightly larger than the outer diameter D26 of the flange 26, with a ratio d34/D26 below 1.05.

In the unstressed configuration, i.e., without outside force applied thereon, the tabs 36 extend at a radial distance d36 from the axis X30 smaller than half the diameter d34. In other words, the tabs 36 protrude radially toward the inside of the volume V32 relative to the rest of the head 34.

Furthermore, and as emerges more particularly from FIGS. 4, 5, 7 and 8, the body 32 bears, on its outer radial surface 324 and near the end 322, two pins 38 protruding radially relative to the surface 324 and that are diametrically opposite. The axial distance d38 between the pins 38 and the end 322 is smaller than 10 mm. Each pin 38 is intended to cooperate with a groove of the base 4, only one of which is shown in FIG. 3 with reference 48. Each groove 48 comprises a longitudinal branch, parallel to the axis X2, and a branch that is transverse or inclined relative to this axis. Thus, the elements 38 and 48 together constitute a bayonet locking system for the housing 30, and thus the assembly 8, on the base 4.

To manufacture each assembly 8, one manufactures a cartridge 20 on the one hand and a housing 30 on the other hand with the specificities mentioned above.

When the cartridge 20 needs to be mounted in the housing 30, one begins by inserting the stopper 40 into the head 34 of the housing 30. More specifically, the head 34 forms a volume V34 for receiving and jamming of the stopper 40, as shown in FIG. 9. The volume V34 is part of the volume V32. The placement of the stopper 40 in the volume V34 is shown by arrows F10 in FIGS. 8 and 9.

Once the stopper 40 is in place in the volume V34, the axes X20 and X30 are aligned and the cartridge 20 is pushed into the volume V32, in the direction of arrow F6 in FIGS. 8 and 9 and through the open end 322, until the flange 26 abuts against the stopper 40. In passing, the flange 26 radially, centrifugally relative to the axis X30, pushes the tabs 36 back toward the outside of the head 34, these tabs being able to move radially away from the axis X30 due to their elasticity. When the surface 262 comes into contact with the stopper 40, the flange 26 has axially exceeded, along the axis X30, the tabs 36, which are then positioned, along this axis, at the neck 24, with their respective edges 362 across from or bearing against the surface 264 of the flange 26. Thus, the cartridge 20 is axially locked in the body 32 of the housing 30, irreversibly.

When the cartridge 20 is thus immobilized in the housing 30 and closed off, on the side of the throat 25, by the stopper 40, a draft product 8′ has been formed of a reservoir-forming assembly 8, as identified above. In this draft product, the opening O20 of the cartridge 20 is not yet closed off by the piston 27 and provides access to the volume V22.

Such a draft product is subject to washing, drying and/or inspection operations, with techniques known in themselves. These operations affect, inter alia, the inner volume of the cartridge 20, which is still empty at this stage.

After these operations, each draft product 8′ is inserted into a recess 102 arranged on a support 100. The support 100 is globally in the form of a rectangular plate and is made from plastic. Each recess 102 has a cylindrical shape with a circular section having a shape complementary to the outer surface 324 of the housing 30. In practice, each recess 102 is formed by a sleeve 104 that extends over one side of a primary web 106 of the support 100. As shown in FIG. 10, the support 100 can be equipped with a relatively large number of recesses 102, in the example sixty-four recesses.

When sixty-four draft products 8′ are mounted on the support 100, they can be manipulated together, in particular using a robot.

In particular, the draft products 8′ and the support 100 can be positioned in a container 200, visible in FIG. 11 and which has a shoulder 202 making it possible to reinforce the support 100 at a distance from the bottom 204 of the container 200 such that the lower ends formed by the heads 34 of the draft products 8′ extend at a nonzero vertical distance d200 from the bottom 204. In other words, the draft products 8′ do not touch the bottom 204, such that they do not risk becoming prematurely worn by rubbing against the bottom during transport.

It is then possible to position, on the container 200, a sealing membrane 300 that is not gas-tight. The container 200 and the elements that it contains may then be made sterile by gas injection, in particular ethylene oxide.

The preceding operations may be done on a same industrial site, in particular the site of a glass worker manufacturing the glass cartridges 20 and to whom the housings 30 and the stoppers 40 are delivered. Alternatively, these operations may be done on the site of a molder manufacturing the housings 30 and to whom the cartridges 20 and the stoppers 40 are delivered.

The container 200 and its sterile contents may then be transported to the site of a pharmaceutical laboratory or the site of a drug packaging company, where the membrane 300 is removed from the container 200 before the support 100 is grasped by a robot, which places it below one or several pipettes 400 for filling the cartridges 20 through the openings O20 of their ends 222 that provide access to the volume V22.

In FIG. 12, a single pipette 400 is shown, while arrow F400 shows the flow of the active ingredient toward the inside of a draft product 8′. In practice, several pipettes can be used at the same time, each of them being positioned above the opening O20 of a cartridge 20 belonging to a draft product 8′ row. Thus, the eight draft products 8′ in a row can be filled at the same time through their respective ends 222, which then constitute the upper ends of the cartridges 20.

When this operation has been carried out, the pipette(s) 400 are laterally offset relative to the draft products 8′, which is shown by arrow F402 in FIG. 12. Next, a piston 27 is engaged in each cartridge 20, through the opening O20 of its open end 222, which is shown by arrow F27. This makes it possible to close off the cartridge 20 and to allow each draft product 8′ to be removed from its recess 102, in which it was vertically retained, without any risk of the injectable product present in the volume V22 flowing.

After the draft product 8′ has been removed from the recess 102, or before this removal, it is possible to affix an identification sticker for the contents of the assembly 8 on the outer radial surface 324 of the housing 30. Alternatively, printing or etching of this surface can be considered.

Because the sticker or identification marking of the assembly 8 is arranged on the surface 322, the housing 30 does not need to be transparent to view the corresponding information. It is, however, advantageous for this housing 30 to be at least partially transparent so as to view the level of injectable product present in the volume V22.

As emerges from FIGS. 6 and 7, the end 222 of the body 22 is withdrawn, inside the body 32. In practice, a distance l8 separates the ends 222 and 322 along the axes X20 and X30. Thus, the end 322 extends, along these axes, past the cartridge 20, which is effectively protected from impacts, in particular when the reservoir-forming assembly 8 is placed on a vertical surface with its distal end 82 oriented upward.

In practice, when an assembly 8 has been used on the injection pen 2, to the point that its cartridge 20 is empty or practically empty, this assembly 8 can be removed from the base 4 via a rotational movement around the axes X2, X20 and X30, then combined, and a translational movement parallel to this axis making it possible to unlock the bayonet system formed by the elements 38 and 48. Next, a new reservoir-forming assembly 8, the cartridge 20 of which is full, can be mounted on the base 4, through a new translation/rotation movement activating the bayonet system.

In this case, the assembly 8 previously disassembled from the injection pen 2 is discarded. In other words, the elements 20 and 30 are disposable after use.

The outer thread 31 is formed on the periphery of the head 34 and makes it possible to mount, successively on the assembly 8 in place on the base 4, disposable elements 10 each time a dose of the product contained in the cartridge 20 must be injected in a patient. The mounting of the disposable element 10 on the distal end 82 of the assembly 8 formed by the head 34 results in perforating the stopper 40 through the opening 344. When the disposable element 10 is in place on the assembly 8, a pushing force, oriented toward the head 34, is exerted on the piston 27 as shown by arrows F8 in FIGS. 6 and 7, in order to drive part of the product present in the inner volume V22 of the body 22 toward the needle 12 of the disposable element 10.

Furthermore, the geometry and positioning of the pins 38 can be configured based on the geometry and positioning of the grooves 48. This makes it possible to pair a type of reservoir-forming element 8 with a type of injection pen 2. One thus avoids the risks of incorrect handling, which would consist of mounting an assembly 8 on an injection pen 2 that is not suitable for said injection pen, for example because the volume V22 has a diameter different from that for which the linear movement of the pushing means on the piston 27 is calculated. In particular, the pins 38 and grooves 48 are not necessarily diametrically opposite.

The thread 31 constitutes a mechanical member for fastening the needle 12 on the housing 30. Alternatively, other types of mechanical members can be considered to perform this function, in particular lugs allowing the disposable element 20 to be mounted via a bayonet system.

Likewise, the pins 38 constitute mechanical members for fastening the housing 30 on the base 4. Alternatively, they can be replaced by other mechanical members performing the same function, for example a thread arranged on the surface 324, as long as the base 4 is equipped with a corresponding tapping. Furthermore, if the body 32 protrudes enough from the body 22 on the side of the proximal end 84 of the element 8, a tapping can be provided on the inner surface of the body 32 while a corresponding thread is provided on the base 4.

In the bayonet system(s) used, the male and female reliefs, i.e., the pins 38 and the grooves 48, are positioned on the housing 30 or on the base 40, as chosen.

According to one alternative of the invention that is not shown, the stopper 40 can be overmolded in the housing 30 during step a). In this case, step d) is not carried out.

The embodiments and alternatives considered above may be combined to generate new embodiments of the invention.

1-15. (canceled)

16. A method for producing at least one assembly forming an injectable product reservoir for an injection pen, this method comprising a step a) consisting of producing a hollow housing equipped with a first mechanical member for fastening a needle and a second mechanical member for fastening the housing on an injection pen, wherein it comprises at least some successive steps, after step a) and consisting of:
b) inserting, in an inner volume of the housing, an elongated container with an open bottom,
c) filling the container with a predetermined quantity of injectable product, through an opening of the open bottom of the container.

17. The method according to claim 16, wherein during step b), the insertion of the container into the hollow housing takes place through an open end of a body of this housing in the vicinity of which the second mechanical member is positioned for fastening the housing on an injection pen.

18. The method according to claim 16, wherein, prior to step b), a longitudinal axis of the container is aligned with a longitudinal axis of the hollow housing and, during step b), the insertion of the container into the housing takes place by a translation of the container relative to the hollow housing along these two longitudinal axes.

19. The method according to claim 16 wherein, during step b), the container is moved inside the housing until a flange of the container is axially immobilized, irreversibly, by at least one third mechanical locking member positioned in the vicinity of the first mechanical member for fastening a needle.

20. The method according to claim 19, wherein the third locking member is an elastically deformable tab of the housing, wherein, during step b), the flange of the container radially pushes the tab back, which deforms elastically, and wherein, when the flange has axially exceeded the tab, the latter regains its original configuration, as a result of which one edge of the tab, oriented opposite an open end of a body of the hollow housing, arrives across from, or bearing against, an annular surface of the flange turned toward one end of a body of the container opposite a throat of this container.

21. The method according to claim 16, wherein it comprises a step d) after step a) and before step b), consisting of:
d) inserting a stopper into the housing for closing a throat of the container.

22. The method according to claim 16, wherein, during step b), the container is fully inserted into the inner volume of the housing.

23. The method according to claim 16, wherein it comprises a step e), after step b) and before step c), consisting of:
e) positioning a draft product, formed from the housing equipped with the container, on a support, with the open bottom of the container oriented upward.

24. The method according to claim 23, wherein it comprises additional steps consisting of:
f) before or after step e), positioning the support in a container,
g) closing the container using a closure member,
h) after step g), sterilizing the draft product.

25. The method according to claim 24, wherein, at the end of step f), the housing extends at a nonzero distance above the bottom of the container.

26. The method according to claim 16, wherein steps a), b) and optionally d), e), f), g) and/or h) are carried out on a same industrial site, in particular a manufacturer of the container and/or the housing.

27. The method according to claim 23 for producing several assemblies forming an injectable product reservoir for an injection pen, wherein the support is equipped with several recesses, each with a shape complementary to the outer shape of a housing, and wherein step e) is carried out successively, in order to have a housing equipped with a container in each recess.

28. The method according to claim 16, wherein it comprises additional steps i) and j) after step c) and consisting of:
i) closing off the open bottom of the container with a piston inserted into an inner volume of this container,
j) identifying each reservoir-forming assembly for an injectable product by affixing a sticker or a marking on an outer surface of the housing,
and wherein steps c), i) and j) are carried out on a same industrial site, in particular within a pharmaceutical laboratory or on a drug packaging site.
